(19) United States
(12) Plant Patent Application Publication
(10) Pub. No.: US 2002/0095697 P1
(43) Pub. Date: Jul. 18, 2002

(54) Shrub rose plant named 'Meipreston'

(76) Inventor: Alain A. Meilland, Antibes (FR)

Correspondence Address: BURNS DOANE SWECKER & MATHIS L L P, POST OFFICE BOX 1404, ALEXANDRIA, VA, 22313-1404, US

(21) Appl. No.: 09/759,263

(22) Filed: Jan. 16, 2001

(51) Int. Cl. A01H 5/00

(52) U.S. Cl. PLT/108

(57) ABSTRACT

A new and distinct variety of landscape shrub rose plant is provided which forms in abundance semi-double blossoms in clusters that are ruby red in coloration. The vegetation is strong and vigorous. A dense and compact mounding shrub growth habit is displayed. Attractive dense medium green foliage with a satin finish is formed. Very good disease resistance is exhibited. Attractive ornamentation in the landscape is provided that is smaller and more compact than that provided by the ‘Meikrotal’ variety (U.S. Plant Pat. No. 6087).

### SUMMARY OF THE INVENTION

The new variety of Rosa hybrida landscape shrub rose plant of the present invention was created by artificial pollination wherein two parents were crossed which previously had been studied in the hope that they would contribute the desired characteristics. The female parent (i.e., the seed parent) of the new variety was the product of the cross of the ‘Meineble’ variety (U.S. Plant Pat. No. 7116) and the ‘Meikrotal’ variety (U.S. Plant Pat. No. 6087). The male parent (i.e., the pollen parent) of the new variety was the ‘The Fairy’ variety (non-patented in the United States). The parentage of the new variety can be summarized as follows:

(‘Meineble’×‘Meikrotal’)×‘The Fairy’.

The seeds resulting from the above pollination were sown and small plants were obtained which were physically and biologically different from each other. Selective study resulted in the identification of a single plant of the new variety.

It was found that the new variety of landscape shrub rose plant of the present invention possesses the following combination of characteristics:

(a) abundantly forms in clusters attractive semi-double blossoms that are ruby red in coloration,

(b) exhibits a compact mounding shrub growth habit,

(c) forms vigorous vegetation,

(d) forms dense medium green foliage with a satin finish, and

(e) exhibits very good disease resistance.

The new variety well meets the needs of the horticultural industry. It can be grown to advantage as attractive dense and compact ornamentation in parks, gardens, public areas, and residential landscapes. It is particularly well suited for growing in the landscape where informal ornamentation is desired. The ruby red blossoms contrast nicely with the medium green foliage.

The new variety of the present invention forms a smaller and more compact plant than its ‘Meikrotal’ parent.

The characteristics of the new variety have been found to be homogeneous and stable and are strictly transmissible at West Grove, Pa. by asexual propagation such as budding, grafting, and cuttage from one generation to another. The plant propagates well on its own roots when propagated from cuttings.

The new variety has been named the ‘Meipreston’ variety. The new variety is being marketed under the RUBY MEIDILAND trademark.

### BRIEF DESCRIPTION OF THE PHOTOGRAPH

The accompanying photograph shows, as nearly true as it is reasonably possible to make the same in a color illustration of this character, typical specimens of the plant and plant parts of the new variety. The rose plants of the new variety illustrated herein were three years of age and were grown outdoors during the fall on their own roots at West Grove, Pa. Dimensions in inches and centimeters are indicated at the right of the photograph.

FIG. 1—illustrates a specimen of a young shoot;

FIG. 2—illustrates a specimen of floral buds before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem with foliage;

FIG. 13—illustrates a specimen of a main branch with foliage;

FIG. 14—illustrates a specimen of a leaf with three leaflets—plan view—upper surface;

FIG. 15—illustrates a specimen of a leaf with five leaflets—plan view—under surface; and

FIG. 16—illustrates specimens of maturing petals.

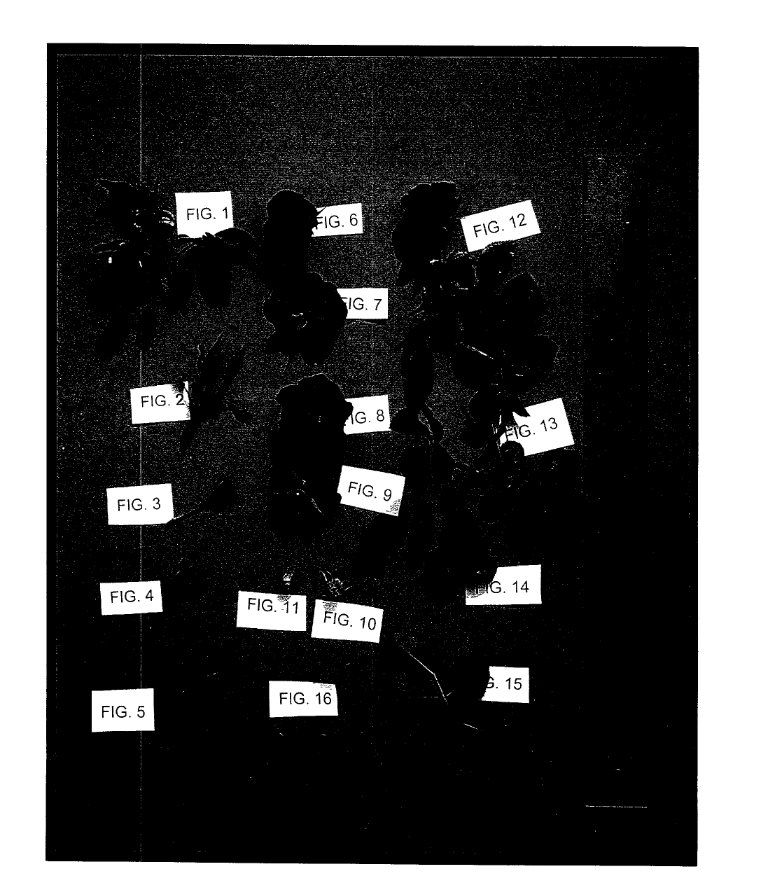

### DETAILED DESCRIPTION

The chart used in the identification of colors is that of The Royal Horticultural Society (R.H.S. Colour Chart). The description is based on the observation of two year-old specimens of the new variety during October while grown outdoors on their own roots at West Grove, Pa.

Class: Landscape Shrub Rose.

Plant:

Height.—approximately 1 meter on average at the end of the growing season.

Width.—approximately ⅔ meter on average at the end of the growing season.

Habit.—compact and mounding.

Branches:

Color.—young stems: Yellow-Green Group 144C with highlights of Red Group 53B. —adult wood: Yellow-Green Group 146B.

Thorns.—shape: needle-shaped as well as some bristles. —size: approximately 5 mm on average. —quantity: moderately numerous, approximately 15 true thorns plus bristles on a stem having a length of 30 cm. —color: Greyed-Purple Group 185B.

Leaves:

Stipules.—narrow and triangular with the auricle facing downward.

Petioles.—upper surface: Yellow-Green Group 144A. —under surface: Yellow-Green Group 144C.

Leaflets.—number: 3, 5, and 7. —shape: ovate with rounded base and acute apices. —serration: regular. —texture: consistent. —overall appearance: dense, medium green in coloration, and leathery with a satin finish. —color (young foliage): upper surface: near Green Group 141B and 141C with highlights of Greyed-Purple Group 184B. under surface: Green Group 141C with highlights of Greyed-Purple Group 184B. —color (adult foliage): upper surface: Green Group 139A. under surface: Yellow-Green Group 146B.

Inflorescence:

Number of flowers.—commonly approximately 1 to 8 and commonly in a cluster.

Peduncle.—Yellow-Green Group 144B with pubescence of White Group 155D, and approximately 3 cm in length on average.

Sepals.—upper surface: Yellow-Green Group 144B with pubescence of White Group 155D. —under surface: Green Group 143B.

Buds.—shape: globose. —length: approximately 2 cm on average. —size: small to medium. —color (when opening): upper surface: Red Group 45B. under surface: Red Group 45A.

Flower.—form: semi-double and cup-shaped. —diameter: approximately 4 cm on average. —color (when opening begins): upper surface: Red Group 46B and 46C. under surface: Red Group 46B. —color (when blooming): upper surface: Red Group 46B and 46C. —under surface: Red Group 46A and 46B. —color (at end of blooming): upper surface: Red Group 46B. under surface: Red Group 46A. —petal shape: elliptic with a notch. —petal number: approximately 10 on average. —stamen number: approximately 70 on average. —anthers: Brown Group 200C in coloration. —filaments: Greyed-Yellow Group 161C in coloration. —pistils: approximately 30 on average. —stigmas: Yellow-White Group 158A in coloration. —styles: Yellow-White Group 158B in coloration. —receptacle: separate and free with achenes on the bottom and the wall.

Development:

Vegetation.—vigorous and strong.

Blossoming.—abundant and everbearing.

Resistance to diseases.—very good.

I claim:

1. A new and distinct variety of landscape shrub rose plant characterized by the following combination of characteristics:
(a) abundantly forms in clusters attractive semi-double blossoms that are ruby red in coloration,
(b) exhibits a compact mounding shrub growth habit,
(c) forms vigorous vegetation,
(d) forms dense medium green foliage with a satin finish, and
(e) exhibits very good disease resistance;
substantially as herein shown and described.
